(19) United States
(12) Patent Application Publication
(10) Pub. No.: US 2025/0299785 A1
(43) Pub. Date: Sep. 25, 2025

(54) MOLECULAR REPRESENTATION METHOD AND ELECTRONIC DEVICE

(71) Applicants: Beijing Youzhuju Network Technology Co., Ltd., Beijing (CN); Lemon Inc., Grand Cayman (KY)

(72) Inventors: Yiqun WANG, Los Angeles, CA (US); Yuning SHEN, Los Angeles, CA (US); Lihao WANG, Beijing (CN); Shi CHEN, Los Angeles, CA (US)

(21) Appl. No.: 18/863,309

(22) PCT Filed: Aug. 28, 2023
(86) PCT No.: PCT/CN2023/115317

§ 371 Date: Nov. 5, 2024

(30) Foreign Application Priority Data
Sep. 21, 2022 (CN) 202211150982.3

(51) Int. Cl. G16C 20/50; G06N 3/049; G06N 3/086; G16C 20/70

(52) U.S. Cl. CPC G16C 20/50; G06N 3/049; G06N 3/086; G16C 20/70

(57) ABSTRACT

Embodiments of the present disclosure relate to a molecular representation method and an electronic device. The molecular representation method comprises: determining a molecular surface of a molecule, the molecular surface being a continuous Riemannian manifold and the molecular surface comprising a plurality of discrete surface nodes; determining a geometric feature of the molecule based on the molecular surface; determining a chemical feature of the molecule by mapping atomic information inside the molecule to the plurality of surface nodes; determining a unified feature of the molecule by integrating the geometric feature and the chemical feature; and determining a time-dependent evolution multi-scale feature of the molecule based on the unified feature by using a time-dependent evolution neural network model.

### CROSS-REFERENCE TO RELATED APPLICATION(S)

The present application claims priority to Chinese Patent Application No. 202211150982.3, filed with the China National Intellectual Property Administration on Sep. 21, 2022, and entitled “MOLECULAR REPRESENTATION METHOD AND ELECTRONIC DEVICE”, which is incorporated herein by reference in its entirety.

### FIELD

The present disclosure generally relates to the field of computers and the field of bioinformation, and more particularly to a molecular representation method and an electronic device.

### BACKGROUND

In recent years, accelerating new drug research and development using artificial intelligence technologies (such as machine learning, deep learning, and the like) has become an important development direction in the field of biopharmaceuticals. Compared with a traditional wet experiment method, such as synthesis of a new drug and testing of the activity of the new drug by an expert in a laboratory, drug research and development based on artificial intelligence can significantly accelerate a new drug research and development rate by means of computer simulation and high-throughput screening. However, artificial intelligence technologies cannot directly act on drug molecules in a laboratory. Instead, the drug molecules need to be characterized by a molecular representation method to achieve computer modeling. Common molecular representation methods include a molecular graph, a point cloud, a three-dimensional voxel, and the like.

However, currently common molecular representation methods cannot fully represent overall information of a molecule. Therefore, a more universal molecular representation method is needed.

### SUMMARY

According to example embodiments of the present disclosure, there is provided a molecular representation method for determining a time-dependent evolution multi-scale feature of a molecule based on a Riemannian manifold of a molecular surface.

In a first aspect of embodiments of the present disclosure, there is provided a molecular representation method, comprising: determining a molecular surface of a molecule, the molecular surface being a continuous Riemannian manifold and the molecular surface comprising a plurality of discrete surface nodes; determining a geometric feature of the molecule based on the molecular surface; determining a chemical feature of the molecule by mapping atomic information inside the molecule to the plurality of surface nodes; determining a unified feature of the molecule by integrating the geometric feature and the chemical feature; and determining a time-dependent evolution multi-scale feature of the molecule based on the unified feature by using a time-dependent evolution neural network model.

In a second aspect of embodiments of the present disclosure, there is provided an electronic device, comprising: at least one processing unit; at least one memory, the at least one memory being coupled to the at least one processing unit and storing instructions executable by the at least one processing unit, the instructions, when executed by the at least one processing unit, causing the electronic device to perform the method according to the first aspect of the present disclosure.

In a third aspect of embodiments of the present disclosure, there is provided a computer-readable storage medium having machine-executable instructions stored thereon, the machine-executable instructions, when executed by a device, causing the device to perform the method according to the first aspect of the present disclosure.

In a fourth aspect of embodiments of the present disclosure, there is provided a computer program product comprising computer-executable instructions, wherein the computer-executable instructions, when executed by a processor, implement the method according to the first aspect of the present disclosure.

In a fifth aspect of embodiments of the present disclosure, there is provided an electronic device, comprising: processing circuitry configured to perform the method according to the first aspect of the present disclosure.

The Summary section is provided to introduce a selection of concepts in a simplified form that are further described below in the Detailed Description section. The Summary section is not intended to identify key features or essential features of the present disclosure, nor is it intended to limit the scope of the present disclosure. Other features of the present disclosure will be readily understood through the following description.

### BRIEF DESCRIPTION OF THE DRAWINGS

The above and other features, advantages, and aspects of the embodiments of the present disclosure will become more apparent with reference to the following detailed description in conjunction with the accompanying drawings. In the drawings, the same or similar reference numerals denote the same or similar elements, where:

FIG. 1 shows a schematic diagram of various different molecular representation methods for a benzene molecule;

FIG. 2 shows a schematic flowchart of an example process according to some embodiments of the present disclosure;

FIG. 3 shows a schematic diagram of an electron density field of a benzene molecule according to some embodiments of the present disclosure;

FIG. 4A and FIG. 4B respectively show schematic diagrams of a molecular surface represented by triangulation according to some embodiments of the present disclosure;

FIG. 5A shows a schematic diagram of projecting chemical information of atoms to a node on a molecular surface according to some embodiments of the present disclosure;

FIG. 5B shows a schematic diagram of an electrostatic potential energy function of a molecular surface according to some embodiments of the present disclosure;

FIG. 6 shows a schematic diagram of a distribution of the first six eigenfunctions of a molecule on a molecular surface according to some embodiments of the present disclosure;

FIG. 7 shows a schematic diagram of a change in a thermal distribution on a molecular surface over time according to some embodiments of the present disclosure;

FIG. 8 shows a schematic diagram of determining a time-dependent evolution multi-scale feature according to some embodiments of the present disclosure;

FIG. 9A and FIG. 9B show schematic diagrams of a spatial relationship between a pair of mirror-symmetric chiral molecules and a set of function gradients corresponding to a surface according to some embodiments of the present disclosure;

FIG. 10 shows a block diagram of an example apparatus according to some embodiments of the present disclosure; and

FIG. 11 shows a block diagram of an example device that can be used to implement embodiments of the present disclosure.

### DETAILED DESCRIPTION OF EMBODIMENTS

Embodiments of the present disclosure will be described in more detail below with reference to the accompanying drawings. Although some embodiments of the present disclosure are shown in the drawings, it should be understood that the present disclosure may be implemented in various forms and should not be construed as being limited to the embodiments set forth herein. Rather, these embodiments are provided for a more thorough and complete understanding of the present disclosure. It should be understood that the accompanying drawings and the embodiments of the present disclosure are only for exemplary purposes, and are not intended to limit the scope of protection of the present disclosure.

As described above, by using artificial intelligence technologies such as machine learning, the activity test of drug molecules and the like can be accelerated. Drug molecules can be characterized by molecular representation methods for quantitative modeling. In the case of a limited number of known molecules, a machine learning model can be used to predict properties of a molecule based on a molecular representation method (for example, a representation method containing rich information of the molecule). However, current molecular representation methods cannot fully represent the information of a molecule. Even though machine learning can learn some features that are not contained in the original representation from big data, in the case of limited data, for example, in most biopharmaceutical problems, a more effective molecular representation method is needed to represent the information of a molecule more fully.

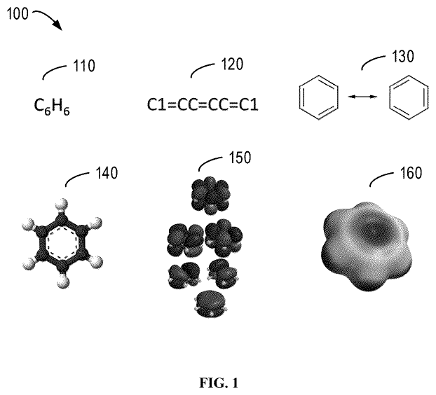

FIG. 1 shows a schematic diagram of various different molecular representation methods for a benzene molecule 100. In FIG. 1, a molecular formula representation 110, a SMILES representation 120, a graph representation 130, a ball-and-stick representation 140, a molecular orbital representation 150, and an electron density field representation 160 are shown. Any of the molecular representation methods 110 to 160 can be used to model the benzene molecule, but the molecular information contained in the different molecular representation methods is different. For example, the molecular formula representation 110 does not contain any three-dimensional structure information. As shown, the graph representation 130 is in a form of a Kekulé structure, although the connection relationship between atoms can be effectively represented, the spatial distribution of its electron cloud, such as the space occupancy of the molecule, is not explicitly expressed.

Although various different molecular representation methods can be respectively used in different scenarios, common molecular representation methods usually do not model a molecule as a whole. Instead, they only model local structure and chemical information. However, actual physical chemistry is multi-scale, for example, an electrostatic force is a long-range interaction, and therefore the current local molecular representation method cannot model more accurately in accordance with physical laws. Moreover, such limitations will cause the corresponding machine learning model to be unable to effectively model the quantitative structure-activity relationship of the molecule, thereby also affecting the success rate of downstream biopharmaceutical tasks.

At least to solve the above problems and other potential problems, embodiments of the present disclosure provide a molecular representation solution. Specifically, a time-dependent evolution multi-scale feature of a molecule is determined based on a Riemannian manifold of a molecular surface to represent chemical information and geometric information of the molecule, so that both local features and overall features of the molecule are included, thus the included information is more comprehensive. The molecular representation method in embodiments of the present disclosure can be used for modeling in artificial intelligence technologies such as machine learning, for example, can more effectively represent the activity of a molecule, thereby improving the success rate of biopharmaceutical tasks.

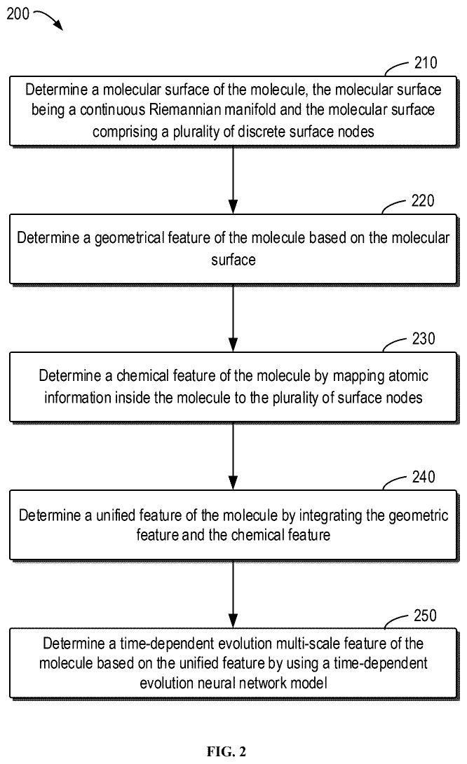

FIG. 2 shows a schematic flowchart of an example process 200 according to some embodiments of the present disclosure. At block 210, a molecular surface of a molecule is determined, the molecular surface is a continuous Riemannian manifold and the molecular surface comprises a plurality of discrete surface nodes. At block 220, a geometric feature of the molecule is determined based on the molecular surface. At block 230, a chemical feature of the molecule is determined by mapping atomic information inside the molecule to the plurality of surface nodes. At block 240, a unified feature of the molecule is determined by integrating the geometric feature and the chemical feature. At block 250, a time-dependent evolution multi-scale feature of the molecule is determined based on the unified feature by using a time-dependent evolution neural network model.

Exemplarily, the molecule in the embodiments of the present disclosure may be a biological macromolecule, such as protein, DNA, etc., or may be a small molecule, such as a small molecule of an aspirin drug, etc. This is not limited in the present disclosure.

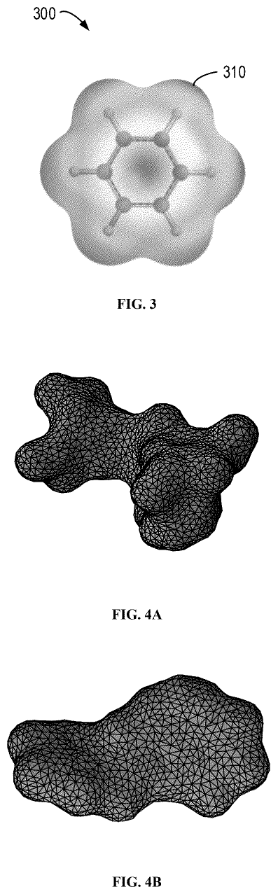

Exemplarily, the embodiments of the present disclosure may determine the chemical feature and the geometric feature based on the Riemannian manifold of the molecular surface. Exemplarily, the geometric feature may be determined based on eigenfunctions and eigenvalues of a Laplace operator. Some embodiments of the present disclosure will be described in more detail below in conjunction with FIGS. 3-10.

In some exemplary embodiments of the present disclosure, the molecular surface of the molecule may be determined based on an isosurface of the electron density field of the molecule.

The scale of biomolecules is generally in the order of 10⁻¹⁰ meters (angstroms). At this microscopic scale, biomolecules generally follow the physical laws described by quantum mechanics and statistical mechanics, rather than Newtonian mechanics at the macroscopic scale. From the perspective of microelectronic structure, a molecule consists of a positively charged nucleus and a negatively charged electron cloud. Intuitively, a molecule can be understood as an electron density field. Different biomolecules have different chemical compositions and three-dimensional geometric structures, thereby exhibiting different physicochemical properties, for example, a specific drug molecule may be combined with a certain protein receptor in a human body to achieve therapeutic effects. That is to say, different molecules have unique electron density fields, so different molecules can be represented by describing the shape and chemical properties of the density field. Specifically, an isosurface of the density field may be determined, which is referred to as the molecular surface of the molecule.

As an example, FIG. 3 shows an electron density field 300 of a benzene molecule according to embodiments of the present disclosure. In FIG. 3, a curve 310 represents the isosurface.

Exemplarily, the electron density field of the molecule may be represented as an electron density function of the molecule. Optionally, the electron density function of the molecule may be determined by means of quantum chemical simulation, and further, the molecular surface may be determined based on an isosurface of the electron density function of the molecule. For example, there may be a plurality of isosurfaces for the electron density function of the molecule, and then in some embodiments of the present disclosure, the molecular surface may be determined by selecting one of the isosurfaces.

In some exemplary embodiments of the present disclosure, the molecular surface may also be determined by other molecular surface calculation methods. For example, the molecular surface of the molecule may be determined by using MSMS calculation software.

In some exemplary embodiments of the present disclosure, the molecular surface of the molecule may also be determined based on sampling of solvent accessible or solvent inaccessible surfaces of the molecule.

It may be understood that in other examples, the molecular surface of the molecule may also be determined in other manners in the embodiments of the present disclosure, which is not limited in the present disclosure.

In some examples, the molecular surface may be represented as a plurality of discrete nodes and a connection relationship between the nodes. Exemplarily, surface information may be further determined based on the determined molecular surface. For example, a grid representation method such as triangulation may be used to store the surface information. FIG. 4A and FIG. 4B show schematic diagrams of a molecular surface represented by triangulation. As shown in the figure, there are triangulation nodes (referred to as “nodes” for short) on the surface, and there may be a connection relationship between the nodes. That is, the molecular surface comprises a plurality of surface nodes, such as a plurality of triangulation nodes.

Exemplarily, the surface wraps the molecule and can express the shape of the molecule. In the embodiments of the present disclosure, the stored surface information may comprise: atomic information inside the molecule, three-dimensional coordinates of each node on the molecular surface, and a connection relationship between the nodes on the molecular surface. For example, the atomic information inside the molecule includes three-dimensional coordinates of the atoms, an atomic type, and other related chemical information. It may be understood that the molecular surface is a two-dimensional Riemannian manifold, and the manifold is continuous and smooth. In the subsequent processing procedure of the embodiments of the present disclosure, the continuous and smooth Riemannian manifold may be discretized, e.g., to the triangulation nodes.

In some exemplary embodiments of the present disclosure, for each of the plurality of surface nodes, a chemical environment feature of the node is obtained by mapping atomic information of a plurality of atoms associated with the node to the node; and the chemical feature is determined using a fully connected neural network based on the chemical environment feature of each of the plurality of surface nodes. Exemplarily, the plurality of atoms associated with the node may comprise: a plurality of atoms within a range of a distance from the node lower than a distance threshold. Alternatively, exemplarily, the plurality of atoms associated with the node comprise: a fixed number of nearest atoms (for example, 8 nearest neighbor atoms) from the node. For example, the atoms may be sorted according to the distance from the node, and the nearest fixed number of (for example, 8) atoms may be determined from the sorted atoms.

Specifically, a chemical potential distribution of the molecular surface may be determined based on the surface information of the molecule. Optionally, the chemical potential distribution may also be referred to as a chemical function distribution, e.g., an electrostatic potential energy distribution.

Exemplarily, for any node on the molecule surface, a distance between all atoms within a specific distance range around the node and the node may be determined. For example, an atom within a distance threshold range may be referred to as a neighboring atom. Subsequently, a normal angle between each neighboring atom and a tangent plane of a curved surface where the node is located, and a corresponding atomic type may be determined, which are used as an initial representation of the chemical environment of the node. Exemplarily, the chemical function distribution of the molecular surface may be extracted by a fully connected neural network. That is, the representation of the chemical environment around the surface node can be learned through the fully connected neural network.

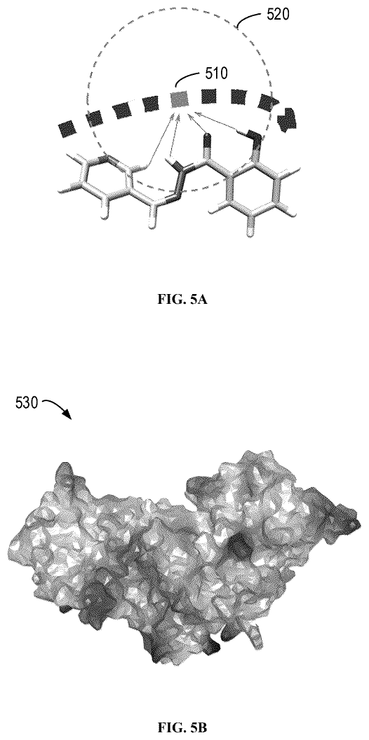

In this way, by mapping (also referred to as projecting) the chemical information of internal atoms to a node on the surface, the chemical information of the entire molecule can be characterized by the node on the molecular surface. FIG. 5A shows a schematic diagram of projecting chemical information of atoms to a node on a molecular surface. As shown in the figure, for a node 510, atoms within a specific distance range 520 may be determined. Subsequently, chemical information of the determined atoms may be projected to the node 510 to determine an initial representation of the chemical environment of the node 510, e.g., a chemical environment feature of the node.

It should be noted that in the embodiments of the present disclosure, the chemical representation of a node on the molecular surface may be updated by using the chemical information of the atoms, but the information of the node will not feedback and change the chemical information of the atoms, that is, the projection belongs to a one-way information transfer relationship. It is different from a graph neural network of a molecule with two-way updating. It may be understood that although the graph neural network can realize long-distance information exchange through graph information transfer, the exchange mechanism is inefficient when there are a large number of nodes (for example, there are usually tens of thousands of nodes in a surface triangulation representation of a molecule). In contrast, in the embodiments of the present disclosure, the processing efficiency of information exchange can be improved through the one-way information transfer relationship from the atomic information to the node.

Exemplarily, through the fully connected neural network, the chemical feature of the molecular surface may be determined based on the chemical environment feature of each of the plurality of surface nodes. Optionally, as an example, the chemical information of an atom may be represented as a multi-dimensional (for example, 5-dimensional) array, and the chemical feature of the surface may be represented as a multi-dimensional (for example, 16-dimensional) array.

FIG. 5B shows a schematic diagram of an electrostatic potential energy function 530 of the molecular surface. For example, the electrostatic potential energy function may be obtained by extraction based on the first-dimensional feature in the chemical feature of, for example, a 16-dimensional array. It may be understood that although FIG. 5B takes the electrostatic potential energy function as an example for illustration, the embodiments of the present disclosure are not limited thereto. For example, a user may customize other chemical information, or may learn other chemical representations through a neural network or the like.

In this way, the chemical potential distribution of the molecular surface may contain both geometric information and chemical information. Exemplarily, the distribution of a chemical potential function such as an electrostatic potential energy function on the molecular surface belongs to the surface Riemannian manifold space representation of the molecule, that is, the chemical information may exist in the form of a function in the surface Riemannian manifold space of the molecule. In other words, in the embodiments of the present disclosure, the surface of the molecule is regarded as a continuous and smooth Riemannian manifold space, and a chemical-related function is defined in the two-dimensional manifold space.

In some exemplary embodiments of the present disclosure, the geometric feature may comprise one or more of the following: a heat kernel signature, a wave kernel signature, Gaussian curvature of the molecular surface, or mean curvature of the molecular surface.

Exemplarily, an eigenfunction (or referred to as a Laplace eigenfunction) and an eigenvalue of a Laplace operator on a molecular surface (Riemannian manifold) may be determined, and the heat kernel signature and/or the wave kernel signature may be determined based on the eigenfunction and the eigenvalue.

Exemplarily, the eigenfunction and the eigenvalue of the Laplace-Beltrami operator on each molecular surface manifold may be determined, which is expressed as formula (1):

$${Δϕ}_{i} = {λ}_{i} {ϕ}_{i} \qquad ( 1 )$$

In formula (1), Δ represents the Laplace operator, which is expressed as formula (2):

$$Δ f = {∇}^{2} f = ∇ \cdot ∇ f = \frac{{\partial }^{2} f}{\partial {x}^{2}} + \frac{{\partial }^{2} f}{\partial {y}^{2}} \qquad ( 2 )$$

In formula (1), Øᵢ represents an i-th eigenfunction, and λᵢ represents an i-th eigenvalue. In formula (2), ∇ represents a gradient operator, and ƒ represents an arbitrary function distributed on the Riemannian manifold. Exemplarily, the eigenfunction may be determined by using a known algorithm (for example, scipy numerical calculation software) or an algorithm to be developed in the future, which is not limited in the present disclosure.

In some examples, the Laplace eigenfunction of each molecular surface manifold and its corresponding eigenvalue are unique and are only related to the shape of the molecule itself, and are not affected by the position and orientation of the molecule in three-dimensional space. Therefore, the eigenfunction of the Riemannian manifold is also called a “shape DNA”. For the surface manifold of each molecule, all its eigenfunctions and eigenvalues can be determined. Exemplarily, the eigenvalues may be further sorted according to the size of the eigenvalues, for example, the eigenvalues may be sorted in ascending order, and then the first k (for example, k=100 or other values) eigenvalues in the sorting are taken, which can reduce the amount of calculation.

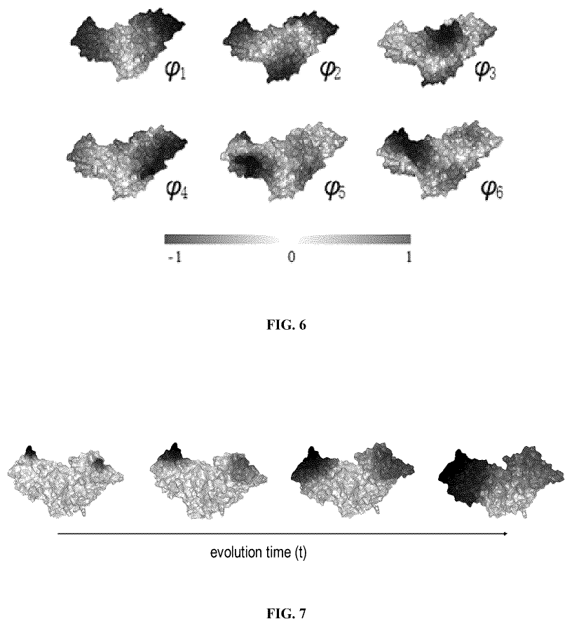

It may be understood that since different biomolecules have different shapes, and thus have different surface manifold eigenfunctions. FIG. 6 shows a distribution of the first six eigenfunctions of a molecule on a molecular surface according to some embodiments of the present disclosure. Exemplarily, the first six eigenfunctions are shown as φ₁-φ₆ in FIG. 6. In some examples, the eigenfunctions show regional undulations in FIG. 6. Correspondingly, the eigenfunction can be understood as a Fourier basis function in a two-dimensional manifold space (for example, it can be understood as a two-dimensional standing wave), which corresponds to a sine function and a cosine function on a one-dimensional straight line.

In some exemplary embodiments of the present disclosure, the geometric feature may be represented in the form of a geometric feature function. A geometric feature function of the molecular surface may be determined based on the eigenfunction and the eigenvalue of the Laplace operator on the molecular surface manifold. Optionally, the geometric feature function may comprise a heat kernel signature (HKS) and/or a wave kernel signature (WKS).

Exemplarily, the HKS and the WKS may be constructed based on the determined eigenfunction Øᵢ and the eigenvalue λᵢ as follows:

$$HKS ( x , t ) = {\sum }_{i} {e}^{- {λ}_{i} t} {ϕ}_{i}^{2} ( x ) \qquad ( 3 )$$

$$WKS ( x , ϵ ) = {\sum }_{k} {ϕ}_{k}^{2} ( x ) {e}^{\frac{- {( ϵ - log {E}_{k} )}^{2}}{2 {σ}^{2}}} \qquad ( 4 )$$

In formulas (3) and (4), t and ϵ respectively represent time and energy, which may be set by the user for example.

Optionally, the geometric feature function of the molecular surface may further comprise Gaussian curvature and/or mean curvature on the molecular surface (Riemannian manifold). It may be understood that the Gaussian curvature and the mean curvature may be obtained through geometric calculation, which will not be repeated here.

In some exemplary embodiments of the present disclosure, a unified feature of the molecule may be determined by integrating the geometric feature and the chemical feature. For example, the geometric feature is represented as a geometric feature function, and the chemical feature is represented as a chemical potential distribution, then the unified feature of the molecular surface may be determined based on the chemical potential distribution of the molecular surface and the geometric feature function of the molecular surface. The unified feature (e.g., represented as a surface feature function) may represent an integration of chemical information and geometric information.

Exemplarily, the chemical feature and the geometric feature of each node may be integrated by a fully connected neural network to obtain a surface feature function on each node. For example, assuming that the chemical feature is represented as a 16-dimensional array, and the geometric feature is represented as a 32-dimensional array, the chemical feature and the geometric feature may be nonlinearly transformed into a 64-dimensional surface feature function by the fully connected neural network. It may be understood that the dimension of the surface feature function is not limited to 64 dimensions, and may be customized by the user, for example, 128 dimensions or other dimensions, which is not limited in the present disclosure.

Exemplarily, the fully connected neural network may be obtained by training based on a molecular dataset, specifically, the molecular dataset is related to an application scenario (e.g., a downstream prediction task) of the embodiments of the present disclosure.

In some exemplary embodiments of the present disclosure, the time-dependent evolution multi-scale feature may be determined based on the unified feature by using the time-dependent evolution neural network model. Exemplarily, the time-dependent evolution multi-scale feature represents a multi-scale feature of the molecular surface.

Exemplarily, the time-dependent evolution neural network model comprises an evolution operator, and the evolution operator is at least based on the Laplace operator and/or a surface potential energy term.

For example, the time-dependent evolution operator may be applied to the surface feature function to obtain a function characterizing the multi-scale feature. For example, the time-dependent evolution operator may be represented as e^{-iĤt} or e^{-Ĥt}, where Ĥ is a Hamiltonian operator, for example, Ĥ=Δ+V, Δ represents the Laplace operator, and V represents the surface potential energy term. For example, the surface potential energy term V may be a function distribution on a manifold set by the user.

In some embodiments, when the time-dependent evolution operator is represented as e^{-iĤt}, for an initial function u₀, the function distribution at time t may be determined by formula (5):

$${u}_{t} = {e}^{- i \hat{H} t} {u}_{0} \qquad ( 5 )$$

To simplify the example, it can be assumed that V=0, so that formula (5) can be simplified to formula (6):

$${u}_{t} = {e}^{- i Δ t} {u}_{0} \qquad ( 6 )$$

Formula (6) describes a change of an initial function u₀ in a manifold space (that is, a molecular surface) over time. By controlling different evolution times t, a new function distribution uₜ after evolution at different times can be obtained. It may be understood that uₜ obtained by formula (6) is a complex number, and the input u₀ is a real number. In practice, the modulus of uₜ may be taken to obtain a real number corresponding to uₜ.

Since different molecules have different geometric structures, their Riemannian manifold spaces are also unique, and the evolution mode of the function u₀ on different manifolds is also determined by the manifold space. Therefore, the evolved function can be used as a new representation of molecular information, and this representation contains the overall and local information of the manifold.

In other embodiments, when the time-dependent evolution operator is represented as e^{-Ĥt} and V=0, for an initial function v₀, the function distribution at time t may be determined by formula (7):

$${v}_{t} = {e}^{- Δ t} {v}_{0} \qquad ( 7 )$$

Formula (7) can be understood as replacing the imaginary time-dependent evolution operator in formula (6) with a real time-dependent evolution operator (removing i). It can be understood that formula (6) belongs to the framework of quantum mechanics, and formula (7) belongs to the framework of classical mechanics. In practical applications, both frameworks can be used to implement the Riemannian manifold representation of the molecule.

In the embodiments of the present disclosure, the initial function u₀ or v₀ may be the aforementioned unified feature, i.e., the surface feature function of the molecule. In this way, the embodiments of the present disclosure may obtain the time-dependent evolution multi-scale feature (that is, uₜ or vₜ) based on the time-dependent evolution operator.

Exemplarily, the time-dependent evolution operator e^{-Δt} in formula (7) may be referred to as a heat operator, which describes a distribution vₜ of an initial thermal distribution v₀ in the manifold space after t time.

As an example, FIG. 7 shows a schematic diagram of a change in a thermal distribution on a molecular surface over time. It may be understood that the change may be quantitatively described by a time-dependent evolution process as shown in formula (7).

It can be seen from FIG. 7 that as the time t becomes larger and larger, the range of heat transfer becomes larger and larger. Therefore, by controlling different evolution times t, multi-scale information transfer (a short time corresponds to small-scale information transfer, and a long time corresponds to large-scale information transfer) can be realized in the Riemannian manifold space of the molecular surface. Therefore, geometric and chemical information of the molecule at different scales can be learned by a neural network based on time-dependent evolution, thereby improving the representation ability of the molecule.

In the embodiments of the present disclosure, the eigenfunction and the eigenvalue of the Laplace operator are described as above in conjunction with formula (1), and therefore, the time-dependent evolution operator may be based on the eigenfunction and the eigenvalue of the Laplace operator on the Riemannian manifold. Based on this, formula (7) may be further expressed as formula (8):

$${v}_{t} = {e}^{- Δ t} {v}_{0} = Φ [ \begin{matrix}{e}^{- {λ}_{0} t} \\ {e}^{- {λ}_{1} t} \\ ⋮\end{matrix} ] ⊙ ( {Φ}^{⊤} {v}_{0} ) \qquad ( 8 )$$

Similarly, formula (6) may be further expressed as formula (9):

$${u}_{t} = {e}^{- Δ t} {u}_{0} = Φ [ \begin{matrix}{e}^{- {λ}_{0} t} \\ {e}^{- {λ}_{1} t} \\ ⋮\end{matrix} ] ⊙ ( {Φ}^{⊤} {u}_{0} ) \qquad ( 9 )$$

In this way, the embodiments of the present disclosure can perform time-dependent evolution in the eigen space by using the Riemannian manifold and its eigenfunction and eigenvalue of the Laplace operator, which is more efficient than the operation in the real space.

As described above, the unified feature may be represented as, for example, a 64-dimensional surface feature function, that is, each node on the molecular surface may be represented by a 64-dimensional array for the unified feature of the node. Then, time-dependent covering may be performed on the 64-dimensional functions respectively based on formula (8) or (9). It may be understood that each function may have its unique evolution time, for example, t may be used as a parameter for the neural network for time-dependent evolution or may be set by the user. After time-dependent evolution, a multi-scale feature on the molecular surface can be obtained, including a series of scale geometric and chemical features.

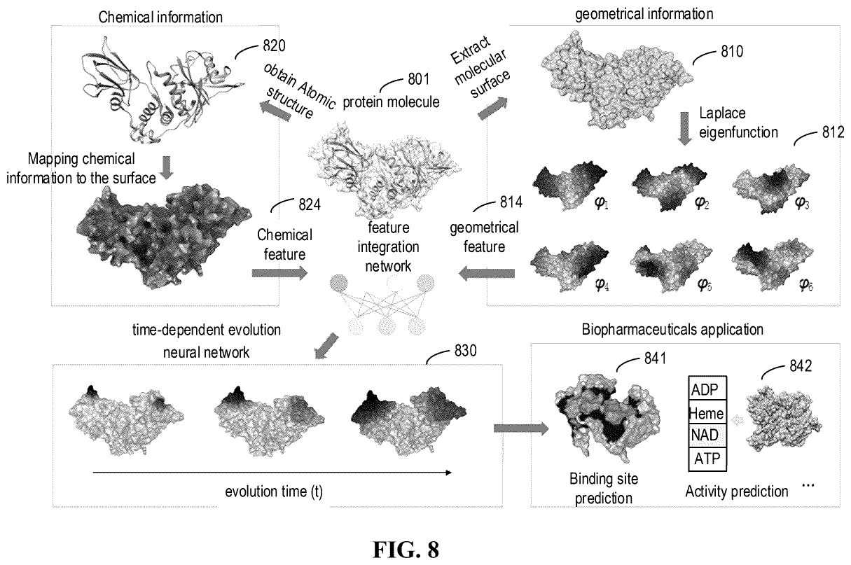

The process 200 of determining the time-dependent evolution multi-scale feature in FIG. 2 is described in more detail above in conjunction with FIGS. 3-7. As an example, FIG. 8 shows a schematic diagram of determining a time-dependent evolution multi-scale feature according to embodiments of the present disclosure. Referring to FIG. 8, for a biomolecule such as a protein molecule 801, a molecular surface 810 may be extracted, and a geometric feature 814 may be obtained by determining an eigenfunction and an eigenvalue 812 of a Laplace operator. A chemical feature 824 may be obtained, based on an atomic structure 820 and the molecular surface 810 obtained from the protein molecule 801, by mapping chemical information to the surface. Further, a unified feature may be obtained based on the geometric feature 814 and the chemical feature 824, for example, through a feature integration network. In addition, a time-dependent evolution multi-scale feature 830 may be further obtained based on a time-dependent evolution neural network. It may be understood that although the protein molecule 801 is taken as an example in FIG. 8, the present disclosure is not limited thereto. In fact, the present disclosure is not limited to the type or size of the molecule.

Additionally or optionally, an overall feature of the molecule may also be determined by average pooling or maximum pooling based on the time-dependent evolution multi-scale feature. This can simplify the representation of the feature.

In the embodiments of the present disclosure, through the time-dependent evolution multi-scale feature, a molecular representation method based on the Riemannian manifold is provided, which is different from the existing molecular representation methods. The time-dependent evolution multi-scale feature comprises the geometric feature and the chemical feature of the molecule, which enhances the description ability of molecular features. It may be understood that although the interaction between molecules in a real system (for example, a human body) is a dynamic process and the configuration of the molecule will constantly change, the molecular representation method of the embodiments of the present disclosure can effectively express different conformations of the molecule.

It may be understood that the molecular representation method in the embodiments of the present disclosure may be applied to downstream biopharmaceuticals. For example, it may be provided to a machine learning model for molecular modeling. Since the present disclosure provides more comprehensive features explicitly to the machine learning model, the learning effect of the machine learning model can be improved, and the machine learning model can better understand the quantitative structure-activity relationship of the molecule and improve the generalization ability of machine learning.

As an example, the solution of the embodiments of the present disclosure can be used to determine a chirality of a mirror-symmetric molecule. Exemplarily, the chirality of the mirror-symmetric molecule may be determined based on a direction gradient of the time-dependent evolution multi-scale feature on the Riemannian manifold.

It may be understood that the representation of the time-dependent evolution multi-scale feature obtained in the embodiments of the present disclosure only contains scalar features and does not have direction (that is, vector) information. However, since the real molecular surface is a two-dimensional Riemannian manifold existing in three-dimensional space, its symmetry needs to be considered in the process of molecular pharmaceutics.

In some embodiments, the mirror-symmetric molecule may be characterized by a direction gradient of the time-dependent evolution multi-scale feature on the Riemannian manifold. That is, the direction gradient of the time-dependent evolution multi-scale feature on the Riemannian manifold is used as a feature of the mirror-symmetric molecule.

Specifically, for any function ƒ, its gradient on the Riemannian manifold can be expressed as formula (10):

$$f ( {v}_{j} ) - f ( {v}_{i} ) = ∇ f \cdot ( {v}_{i} - {v}_{j} ) \qquad ( 10 )$$

In formula (10), vᵢ and vⱼ respectively represent two different nodes of the molecular surface, ƒ(vᵢ) and ƒ(vⱼ) respectively represent function values at the two different nodes, ∇ is the gradient operator, and ∇ƒ is a gradient of the function ƒ on the manifold.

Based on formula (10), if Aᵢ=vⱼ−vᵢ and Dᵢ=ƒ(vⱼ)−ƒ(vᵢ) are defined, then the gradient of the function can be obtained by formula (11):

$$∇ f ( {v}_{i} ) = {( {A}_{i}^{⊤} {A}_{i} )}^{⊤} {A}_{i}^{⊤} {D}_{i} \qquad ( 11 )$$

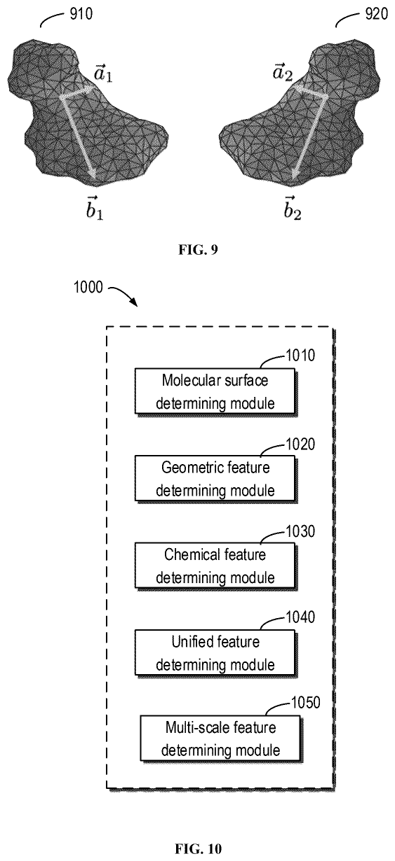

For a pair of mirror-symmetric chiral molecules, their respective function gradient vectors can be obtained through formula (11). FIG. 9 shows a schematic diagram of a spatial relationship between a pair of mirror-symmetric chiral molecules and a set of function gradients corresponding to a surface. Based on this, it can be determined that

$${\vec{a}}_{1} \times {\vec{b}}_{1} = - {\vec{a}}_{2} \times {\vec{b}}_{2} \qquad ( 12 )$$

It can be seen that for a pair of mirror-symmetric molecules, the vector products corresponding to a group of vectors have opposite directions (one points into the paper, and the other points out of the paper). Therefore, the embodiments of the present disclosure may determine the chirality of the mirror-symmetric molecule based on the direction gradient. It may be understood that distinguishing different chiralities is crucial for biopharmaceuticals. For example, some specific chiral drug molecules are active, but different chiral molecules with mirror structures may be harmful to health, such as thalidomide. Therefore, in the embodiments of the present disclosure, the chirality is distinguished by the direction gradient, which can facilitate screening of active molecules in the process of biopharmaceuticals.

Additionally or optionally, information of the direction gradient may be combined with the aforementioned time-dependent evolution multi-scale feature to represent a feature function of the molecular surface. Further, information related to molecules with different chiralities can be learned by a neural network, thereby improving the success rate of downstream pharmaceutic tasks.

As another example, the solution of the embodiments of the present disclosure can be used to determine a binding site of a protein molecule. Exemplarily, at least one of a plurality of surface nodes of the molecular surface may be determined based on the time-dependent evolution multi-scale feature, and the at least one node indicates a site for binding to a virus.

For example, a first region of the molecular surface (for example, a partial region or an entire region of the molecular surface) may be obtained. For at least two nodes in the first region, whether each node can bind to a specific virus may be analyzed, thereby realizing binary prediction. Illustratively, 841 in FIG. 8 shows a schematic diagram of site binding. For example, the molecule may be an antibody protein. Through the analysis of the binding site, the process of drug research and development for resisting viruses can be accelerated.

As a further example, the solution of the embodiments of the present disclosure can be used to determine a biological activity of a molecule. Exemplarily, a target region of the molecular surface may be obtained; a regional time-dependent evolution multi-scale feature corresponding to the target region of the molecular surface may be determined based on the time-dependent evolution multi-scale feature; and at least one predetermined molecule associated with the target region is determined from a plurality of predetermined molecules based on the regional time-dependent evolution multi-scale feature.

For example, a second region of the molecular surface may be obtained as the target region. For a plurality of predetermined molecules, a binding characteristic between the molecule in the target region and the predetermined molecule may be determined. For example, a predetermined molecule with an optimal binding characteristic may be located, and the biological activity of the molecule may be determined based on the located predetermined molecule. Illustratively, 842 in FIG. 8 shows a schematic diagram of biological activity determination, where the plurality of predetermined molecules comprise: adenosine diphosphate (ADP), heme (Heme), nicotinamide adenine dinucleotide (NAD), and adenosine triphosphate (ATP).

It should be noted that although the application in biopharmaceuticals is described above as an example with the binding site and the activity analysis, the embodiments of the present disclosure are not limited thereto. In fact, the molecular representation method based on the Riemannian manifold of the present disclosure can be used in a plurality of applications based on artificial intelligence technologies, which will not be listed here one by one.

It should be understood that in the embodiments of the present disclosure, “first”, “second”, “third”, etc. are only used to indicate that a plurality of objects may be different, but at the same time do not exclude that the two objects are the same, and should not be construed as any limitation to the embodiments of the present disclosure.

It should also be understood that the division of methods, situations, categories, and embodiments in the embodiments of the present disclosure is only for the convenience of description, and should not constitute a special limitation. The features in various methods, categories, situations, and embodiments may be combined with each other under the condition of conforming to the logic.

It should also be understood that the above description is only to help those skilled in the art better understand the embodiments of the present disclosure, but not to limit the scope of the embodiments of the present disclosure. Those skilled in the art can make various modifications, changes, or combinations based on the above content. Such a modified, changed or combined solution is also within the scope of the embodiments of the present disclosure.

It should also be understood that the description of the above content focuses on emphasizing the differences between the various embodiments, and the same or similar parts can refer to each other or draw lessons from each other. For the sake of brevity, they will not be repeated here.

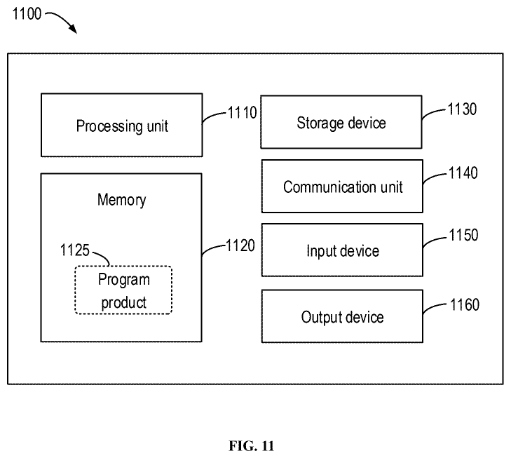

FIG. 10 shows a schematic block diagram of an example apparatus 1000 according to some embodiments of the present disclosure. The apparatus 1000 may be implemented by software, hardware, or a combination of both. As shown in FIG. 10, the apparatus 1000 includes a molecular surface determining module 1010, a geometric feature determining module 1020, a chemical feature determining module 1030, a unified feature determining module 1040, and a multi-scale feature determining module 1050.

The molecular surface determining module 1010 is configured to determine a molecular surface of a molecule, where the molecular surface is a continuous Riemannian manifold and the molecular surface comprises a plurality of discrete surface nodes. The geometric feature determining module 1020 is configured to determine a geometric feature of the molecule based on the molecular surface. The chemical feature determining module 1030 is configured to determine a chemical feature of the molecule by mapping atomic information inside the molecule to the plurality of surface nodes. The unified feature determining module 1040 is configured to determine a unified feature of the molecule by integrating the geometric feature and the chemical feature. The multi-scale feature determining module 1050 is configured to determine a time-dependent evolution multi-scale feature of the molecule based on the unified feature by using a time-dependent evolution neural network model.

In some embodiments, the molecular surface determining module 1010 may be configured to determine the molecular surface based on an isosurface of an electron density field of the molecule.

In some embodiments, the geometric feature comprises a heat kernel signature and/or a wave kernel signature, and the geometric feature determining module 1020 includes: an eigenfunction and eigenvalue determining submodule, configured to determine an eigenfunction and an eigenvalue of a Laplace operator of the molecular surface; a heat kernel signature determining submodule, configured to determine the heat kernel signature based on the eigenfunction and the eigenvalue; and/or a wave kernel signature determining submodule, configured to determine the wave kernel signature based on the eigenfunction and the eigenvalue.

Optionally, the geometric feature determining module 1020 may be configured to: determine Gaussian curvature and/or mean curvature of the molecular surface, and the geometric feature comprises the Gaussian curvature and/or the mean curvature.

In some embodiments, the chemical feature determining module 1030 is configured to: for each of the plurality of surface nodes, obtain a chemical environment feature of the node by mapping atomic information of a plurality of atoms associated with the node to the node; and determine the chemical feature using a fully connected neural network based on the chemical environment feature of each of the plurality of surface nodes.

Optionally, the plurality of atoms associated with the node comprise: a plurality of atoms within a range of a distance from the node lower than a distance threshold.

Exemplarily, the time-dependent evolution neural network model comprises an evolution operator, and the evolution operator is determined based on at least one of the following: an eigenfunction of a Laplace operator on the Riemannian manifold, or a surface potential energy term. Optionally, the surface potential energy term is a function distribution on the Riemannian manifold set by a user.

In some examples, the molecule is a mirror-symmetric molecule, and the apparatus 1000 may further include a chirality determining module, configured to determine a chirality of the mirror-symmetric molecule based on a direction gradient of the time-dependent evolution multi-scale feature on the Riemannian manifold.

In some examples, the molecule comprises a protein molecule, and the apparatus 1000 may further include a site determining module, configured to determine at least one of a plurality of surface nodes of the molecular surface based on the time-dependent evolution multi-scale feature, the at least one node indicating a site for binding to a virus.

In some examples, the apparatus 1000 may further include an activity determining module, configured to: obtain a target region of the molecular surface; determine a regional time-dependent evolution multi-scale feature corresponding to the target region of the molecular surface based on the time-dependent evolution multi-scale feature; and determine at least one predetermined molecule associated with the target region from a plurality of predetermined molecules based on the regional time-dependent evolution multi-scale feature.

In some examples, the apparatus 1000 may further include an overall feature determining module, configured to determine an overall feature of the molecule by average pooling based on the time-dependent evolution multi-scale feature.

The apparatus 1000 of FIG. 10 can be used to implement the process described above in conjunction with FIGS. 2-9. For the sake of brevity, details are not described herein again.

In the embodiments of the present disclosure, the division of the modules or units is schematic, which is only a logical function division, and there may be another division manner in actual implementation. In addition, each functional unit in the disclosed embodiments may be integrated into one unit, or may exist physically separately, or two or more units may be integrated into one unit. The above-mentioned integrated unit may be implemented in the form of hardware, or may be implemented in the form of a software functional unit.

FIG. 11 shows a block diagram of an example device 1100 that can be used to implement the embodiments of the present disclosure. It should be understood that the device 1100 shown in FIG. 11 is merely an example, and should not constitute any limitation to the functions and scope of the implementations described herein. For example, the device 1100 may be used to perform the process described above in conjunction with FIGS. 2-9. For example, the device 1100 may be implemented as a classic computer and/or a quantum computer.

As shown in FIG. 11, the device 1100 is in the form of a general-purpose computing device. Components of the computing device 1100 may include, but are not limited to, one or more processors or processing units 1110, a memory 1120, a storage device 1130, one or more communication units 1140, one or more input devices 1150, and one or more output devices 1160. The processing unit 1110 may be a physical or virtual processor and can execute various processes according to programs stored in the memory 1120. In a multi-processor system, a plurality of processing units execute computer-executable instructions in parallel to improve the parallel processing capability of the computing device 1100.

The computing device 1100 generally includes a plurality of computer storage media. Such media may be any available medium accessible by the computing device 1100, including but not limited to volatile and non-volatile media, detachable and non-detachable media. The memory 1120 may be a volatile memory (for example, a register, a cache, a random access memory (Random Access Memory, RAM)), a non-volatile memory (for example, a read-only memory (Read Only Memory, ROM), an electrically erasable programmable read-only memory (Electrically Erasable Programmable Read Only Memory, EEPROM), a flash memory), or a combination thereof. The storage device 1130 may be a detachable or non-detachable medium, and may include a machine-readable medium, such as a flash drive, a disk, or any other medium, which may be able to be used to store information and/or data (for example, training data for training) and can be accessed in the computing device 1100.

The computing device 1100 may further include another detachable/non-detachable, volatile/non-volatile storage medium. Although not shown in FIG. 11, a disk drive for reading from or writing to a detachable, non-volatile disk (for example, a “floppy disk”) and an optical disk drive for reading from or writing to a detachable, non-volatile optical disk may be provided. In these cases, each drive may be connected to a bus (not shown) through one or more data medium interfaces. The memory 1120 may include a computer program product 1125, which has one or more program modules, and these program modules are configured to execute various methods or actions of various implementations of the present disclosure.

The communication unit 1140 implements communication with other computing devices through a communication medium. Additionally, the functions of the components of the computing device 1100 may be implemented by a single computing cluster or multiple computing machines, and these computing machines can communicate through a communication connection. Therefore, the computing device 1100 may operate in a networked environment using a logical connection with one or more other servers, a network personal computer (Personal Computer, PC), or another network node.

The input device 1150 may be one or more input devices, such as a mouse, a keyboard, a trackball, etc. The output device 1160 may be one or more output devices, such as a display, a speaker, a printer, etc. The computing device 1100 may also communicate with one or more external devices (not shown) as required through the communication unit 1140, for example, a storage device, a display device, etc., communicate with one or more devices that enable a user to interact with the computing device 1100, or communicate with any device (for example, a network card, a modem, etc.) that enables the computing device 1100 to communicate with one or more other computing devices. Such communication may be performed via an input/output (Input/Output, I/O) interface (not shown).

According to an exemplary implementation of the present disclosure, a computer-readable storage medium is provided, having computer-executable instructions stored thereon, where the computer-executable instructions are executed by a processor to implement the method described above. According to an exemplary implementation of the present disclosure, a computer program product is further provided, which is tangibly stored on a non-transitory computer-readable medium and includes computer-executable instructions, and the computer-executable instructions are executed by a processor to implement the method described above. According to an exemplary implementation of the present disclosure, a computer program product is provided, having a computer program stored thereon, where the program, when executed by a processor, implements the method described above.

Various aspects of the present disclosure are described herein with reference to flowcharts and/or block diagrams of a method, an apparatus, a device, and a computer program product implemented according to the present disclosure. It should be understood that each block of the flowchart and/or the block diagram and a combination of blocks in the flowchart and/or the block diagram may be implemented by computer-readable program instructions.

These computer-readable program instructions may be provided to a processing unit of a general-purpose computer, a special-purpose computer, or another programmable data processing apparatus to produce a machine, such that when these instructions are executed through the processing unit of the computer or another programmable data processing apparatus, a device for implementing the functions/actions specified in one or more blocks in the flowchart and/or the block diagram is produced. These computer-readable program instructions may also be stored in a computer-readable storage medium, where the instructions enable a computer, a programmable data processing apparatus, and/or another device to work in a specific manner, so that the computer-readable medium storing the instructions includes a product manufactured, which includes instructions for implementing various aspects of the functions/actions specified in one or more blocks in the flowchart and/or the block diagram.

The computer-readable program instructions may be loaded onto a computer, another programmable data processing apparatus, or another device, such that a series of operation steps are performed on the computer, another programmable data processing apparatus, or another device to produce a computer-implemented process, such that the instructions executed on the computer, another programmable data processing apparatus, or another device implement the functions/actions specified in one or more blocks in the flowchart and/or the block diagram.

The flowcharts and block diagrams in the accompanying drawings show possibly implemented architecture, functions, and operations of the system, the method, and the computer program product according to a plurality of implementations of the present disclosure. In this regard, each block in the flowchart or the block diagram may represent a module, program segment, or part of an instruction, and the module, program segment, or part of the instruction contains one or more executable instructions for implementing a specified logical function. In some alternative implementations, the functions marked in the blocks may also occur in an order different from that marked in the accompanying drawings. For example, two consecutive blocks may actually be executed substantially in parallel, or they may sometimes be executed in a reverse order, depending on a function involved. It should also be noted that each block in the block diagram and/or the flowchart, and a combination of blocks in the block diagram and/or the flowchart may be implemented by a dedicated hardware-based system that executes specified functions or actions, or may be implemented by a combination of dedicated hardware and computer instructions.

The foregoing has described various implementations of the present disclosure. The foregoing description is exemplary, not exhaustive, and is not limited to the disclosed implementations. Many modifications and variations are obvious to a person of ordinary skill in the art without departing from the scope and spirit of the described implementations. The selection of terms used herein is intended to best explain principles of the implementations, practical application, or improvement to technologies in the market, or to enable a person of ordinary skill in the art to understand the implementations disclosed herein.

1. A molecular representation method, comprising:
determining a molecular surface of a molecule, the molecular surface being a continuous Riemannian manifold and the molecular surface comprising a plurality of discrete surface nodes;
determining a geometric feature of the molecule based on the molecular surface;
determining a chemical feature of the molecule by mapping atomic information inside the molecule to the plurality of surface nodes;
determining a unified feature of the molecule by integrating the geometric feature and the chemical feature; and
determining a time-dependent evolution multi-scale feature of the molecule based on the unified feature by using a time-dependent evolution neural network model.

2. The method of claim 1, wherein determining the molecular surface comprises:
determining the molecular surface based on an isosurface of an electron density field of the molecule; or
determining the molecular surface based on sampling of solvent-accessible or inaccessible surfaces of the molecule.

3. The method of claim 1, wherein the geometric feature comprises a heat kernel signature and/or a wave kernel signature, and wherein determining the geometric feature comprises:
determining an eigenfunction and an eigenvalue of a Laplace operator of the molecular surface;
determining the heat kernel signature based on the eigenfunction and the eigenvalue; and/or
determining the wave kernel signature based on the eigenfunction and the eigenvalue.

4. The method of claim 1, wherein determining the geometric feature comprises:
determining Gaussian curvature and/or mean curvature of the molecular surface, and the geometric feature comprises the Gaussian curvature and/or the mean curvature.

5. The method of claim 1, wherein determining the chemical feature comprises:
for each of the plurality of surface nodes, obtaining a chemical environment feature of the node by mapping atomic information of a plurality of atoms associated with the node to the node; and
determining the chemical feature using a fully connected neural network based on the chemical environment feature of each of the plurality of surface nodes.

6. The method of claim 5, wherein the plurality of atoms associated with the node comprise:
a plurality of atoms within a range of a distance from the node lower than a distance threshold; or
a fixed number of plurality of atoms nearest to the node.

7. The method of claim 1, wherein the time-dependent evolution neural network model comprises an evolution operator, and the evolution operator is determined based on at least one of the following:
an eigenfunction of a Laplace operator on the Riemannian manifold, or
a surface potential energy term.

8. The method of claim 7, wherein the surface potential energy term is a function distribution on the Riemannian manifold set by a user.

9. The method of claim 1, wherein the molecule is a mirror-symmetric molecule, and the method further comprises:
determining a chirality of the mirror-symmetric molecule based on a direction gradient of the time-dependent evolution multi-scale feature on the Riemannian manifold.

10. The method of claim 1, wherein the molecule comprises a protein molecule, and the method further comprises:
determining at least one of the plurality of surface nodes of the molecular surface based on the time-dependent evolution multi-scale feature, the at least one node indicating a site for binding to a virus.

11. The method of claim 1, further comprising:
obtaining a target region of the molecular surface;
determining a regional time-dependent evolution multi-scale feature corresponding to the target region of the molecular surface based on the time-dependent evolution multi-scale feature; and
determining, from a plurality of predetermined molecules, at least one predetermined molecule associated with the target region based on the regional time-dependent evolution multi-scale feature.

12. The method of claim 1, further comprising:
determining an overall feature of the molecule by average pooling or maximum pooling based on the time-dependent evolution multi-scale feature.

13. An electronic device, comprising:
at least one processing unit;
at least one memory, the at least one memory being coupled to the at least one processing unit and storing instructions for execution by the at least one processing unit, the instructions, when executed by the at least one processing unit, causing the electronic device to perform actions comprising:
determining a molecular surface of a molecule, the molecular surface being a continuous Riemannian manifold and the molecular surface comprising a plurality of discrete surface nodes;
determining a geometric feature of the molecule based on the molecular surface;
determining a chemical feature of the molecule by mapping atomic information inside the molecule to the plurality of surface nodes;
determining a unified feature of the molecule by integrating the geometric feature and the chemical feature; and
determining a time-dependent evolution multi-scale feature of the molecule based on the unified feature by using a time-dependent evolution neural network model.

14. (canceled)

15. A non-transitory computer-readable storage medium, having a computer program stored thereon, wherein the program, when executed by a processor, implements the method comprising:
determining a molecular surface of a molecule, the molecular surface being a continuous Riemannian manifold and the molecular surface comprising a plurality of discrete surface nodes;
determining a geometric feature of the molecule based on the molecular surface;
determining a chemical feature of the molecule by mapping atomic information inside the molecule to the plurality of surface nodes;
determining a unified feature of the molecule by integrating the geometric feature and the chemical feature; and
determining a time-dependent evolution multi-scale feature of the molecule based on the unified feature by using a time-dependent evolution neural network model.

16. The non-transitory computer-readable storage medium of claim 15, wherein determining the molecular surface comprises:
determining the molecular surface based on an isosurface of an electron density field of the molecule; or
determining the molecular surface based on sampling of solvent-accessible or inaccessible surfaces of the molecule.

17. The non-transitory computer-readable storage medium of claim 15, wherein the geometric feature comprises a heat kernel signature and/or a wave kernel signature, and wherein determining the geometric feature comprises:
determining an eigenfunction and an eigenvalue of a Laplace operator of the molecular surface;
determining the heat kernel signature based on the eigenfunction and the eigenvalue; and/or
determining the wave kernel signature based on the eigenfunction and the eigenvalue.

18. The non-transitory computer-readable storage medium of claim 15, wherein determining the geometric feature comprises:
determining Gaussian curvature and/or mean curvature of the molecular surface, and the geometric feature comprises the Gaussian curvature and/or the mean curvature.

19. The non-transitory computer-readable storage medium of claim 15, wherein determining the chemical feature comprises:
for each of the plurality of surface nodes, obtaining a chemical environment feature of the node by mapping atomic information of a plurality of atoms associated with the node to the node; and
determining the chemical feature using a fully connected neural network based on the chemical environment feature of each of the plurality of surface nodes.

20. The non-transitory computer-readable storage medium of claim 15, wherein the molecule is a mirror-symmetric molecule, and the method further comprises:
determining a chirality of the mirror-symmetric molecule based on a direction gradient of the time-dependent evolution multi-scale feature on the Riemannian manifold.

21. The non-transitory computer-readable storage medium of claim 15, wherein the molecule comprises a protein molecule, and the method further comprises:
determining at least one of the plurality of surface nodes of the molecular surface based on the time-dependent evolution multi-scale feature, the at least one node indicating a site for binding to a virus.
